(19) United States
(12) Patent Application Publication
(10) Pub. No.: US 2023/0228630 A1
(43) Pub. Date: Jul. 20, 2023

(54) SHEAR AND NORMAL FORCE SENSORS, AND SYSTEMS AND METHODS USING THE SAME

(71) Applicant: Purdue Research Foundation, West Lafayette, IN (US)

(72) Inventors: Dane Richter Rudy, Hudson, WI (US); Justin James Markel, South Elgin, IL (US); Jennifer Lorraine Regard, Algonquin, IL (US); Quinton James Lasko, Murfreesboro, TN (US); Christopher Robert Spiers, Glen Ellyn, IL (US)

(73) Assignee: Purdue Research Foundation, West Lafayette, IN (US)

(21) Appl. No.: 17/931,979

(22) Filed: Sep. 14, 2022

Related U.S. Application Data
(63) Continuation of application No. 16/140,816, filed on Sep. 25, 2018, now abandoned.
(60) Provisional application No. 62/563,296, filed on Sep. 26, 2017.

(51) Int. Cl. G01L 1/14; A61B 5/00; G01L 5/165; A61B 5/103; A43B 3/34

(52) U.S. Cl. CPC G01L 1/146; A61B 5/6807; G01L 5/165; A61B 5/1038; A43B 3/34; A61B 2562/0247

(57) ABSTRACT

Sensors capable of sensing shear and normal forces and suitable for measuring reaction forces on a body region of an individual, and systems and methods. Such a sensor includes a first plate and multiple second plates that are separated from the first plate by a dielectric material to define multiple capacitor units that are each responsive to normal and shear forces applied to the sensor. Each capacitor unit comprises an individual second plate of the second plates and a portion of the first plate that is superimposed by the individual second plate. The second plates are superimposed on the first plate so that a shear force applied to the sensor causes a first portion of at least one of the second plates to not be superimposed on the first plate while a remaining portion of the second plate remains superimposed on the first plate to define a superimposed area therebetween.

### CROSS REFERENCE TO RELATED APPLICATIONS

This is a continuation patent application of co-pending U.S. patent application Ser. No. 16/140,816 filed Sep. 25, 2018, which claims the benefit of U.S. Provisional Application No. 62/563,296 filed Sep. 26, 2017. The contents of these prior patent documents are incorporated herein by reference.

### BACKGROUND OF THE INVENTION

The present invention generally relates to sensors and particularly relates to capacitive sensors capable of sensing shear and normal forces, and in so doing finds uses that include but are not limited to measuring reaction forces on a foot.

The current state of modern sports medicine enables the treatment of a multitude of injuries that occur in both contact and non-contact incidents, a significant number of which are non-contact injuries to the leg and foot. Unfortunately, there remains a large void in prediction techniques that could potentially assist in reducing the incidence of non-contact injuries.

One approach to better assessing existing injuries and potential risks is to accurately measure shear and normal forces on the human body. The ability to monitor forces exerted on the bottom of the foot would be extremely useful to address the significant number of non-contact sports injuries that occur due to a limited capability to measure reaction forces that often lead to an injury. Sensors suitable for this purpose must be mobile, compact, and preferably nonintrusive. Current sensors offering an acceptable level of accurate measurements confine the individual to a lab environment, and even then are unable to provide as much information as would be desired to assess existing injuries and potential injury risks.

Force plates are currently the industry standard for accurately collecting three-dimensional (3D) force data. Unfortunately force plates require the individual to perform movements in a lab setting in a very small area. Because of this, force plates are not well suited for thoroughly evaluating potential injury risks to an individual's foot and leg. Though mobile wearable devices exist, many such devices are believed to only measure pressure and do not collect shear force data. Still other devices measure shear forces to monitor 3-D forces, but are cumbersome and difficult to use in any daily application.

In view of the above, it can be appreciated that it would be desirable if a sensing system were available that is capable of measuring 3D forces including shear forces, and utilizes a sensor that can be integrated into apparel (for example, shoes) worn by an individual.

### BRIEF DESCRIPTION OF THE INVENTION

The present invention provides sensors capable of sensing shear and normal forces, and are suitable for measuring reaction forces on a body region of an individual, a nonlimiting example of which is an individual's foot.

According to one aspect of the invention, a sensor for measuring normal and shear forces includes a first plate and multiple second plates that are separated from the first plate by a dielectric material to define multiple capacitor units that are each responsive to normal and shear forces applied to the sensor. Each capacitor unit comprises an individual second plate of the second plates and a portion of the first plate that is superimposed by the individual second plate. The second plates are superimposed on the first plate so that a shear force applied to the sensor causes a first portion of at least one of the second plates to not be superimposed on the first plate while a remaining portion of the second plate remains superimposed on the first plate to define a superimposed area therebetween.

Other aspects of the invention include sensing systems comprising a sensor having aspects as described above, and methods of using a sensor having aspects as described above.

Technical aspects of the sensors, sensing systems, and methods described above preferably include the ability of the sensors to be sufficiently compact to enable the sensors to be integrated into apparel, (for example, shoes) worn by an individual during a physical activity.

Other aspects and advantages of this invention will be further appreciated from the following detailed description.

### BRIEF DESCRIPTION OF THE DRAWINGS

FIG. 1 is a schematic plan view representing capacitive plates of a sensor in accordance with a nonlimiting embodiment of this invention.

FIG. 2 schematically represents two sensors, each configured with capacitive plates as shown in FIG. 1 and aligned for embedment in a shoe insole.

FIG. 3 schematically represents the behavior of a sensor of the type represented in FIGS. 1 and 2 in response to the application of a normal (z-axis) force.

FIG. 4 schematically represents the behavior of the sensor of FIG. 3 in response to the application of a shear force in an X-Y plane of the sensor.

FIGS. 5 and 6 schematically represent the behavior of a sensor of the type represented in FIGS. 1 and 2 in response to multidirectional forces acting in the X-Y plane but not along the X or Y axis.

FIGS. 7A and 7B contain graphs representing calibration results that evidence a very strong correlation between applied normal and shear forces and the deformation of a sensor configured as shown in FIGS. 1 and 2.

### DETAILED DESCRIPTION OF THE INVENTION

The drawings schematically represent sensors capable of measuring 3-D forces. The sensors are particularly adapted for measuring normal and shear forces to which an individual is subject while undergoing physical activities without being obtrusive to the individual. The sensors will be described below in reference to measuring forces to which the human foot is subject, though it should be understood that other applications are within the scope of the invention, including measuring forces experienced by other parts of the human anatomy, living beings other than humans, and nonliving objects.

Particular but nonlimiting embodiments of the sensors will be described below as adapted to be placed within the shoes of a user to monitor forces on the foot as the user exercises, trains, competes, or participates in other sports-related physical activities under normal conditions, during which time the sensors are able to collect data that can be ultimately used to analyze the user's performance, as well as used as a training aid to understand ways to avoid non-contact injuries. Whereas prior sensors ordinarily measure normal forces (or pressure) and neglect shear forces, sensors described herein are capable of providing a more complete 3-D force monitoring capability that better encompasses forces associated with physiological activities, including enabling the monitoring of shear forces as a vital component of a kinetic chain modeled to analyze forces throughout a user's foot and leg. Furthermore, the sensors are compact and capable of being placed or integrated into a user's apparel, for example, embedded within the insole of the user's shoes. For example, the sensors and their electrical hardware can be embedded within a silicone insole, and in so doing are impervious to water, dust, and wear. The sensors can be placed in essentially any type of footwear that might be worn during a physical activity, including sports-related and occupation-related footwear.

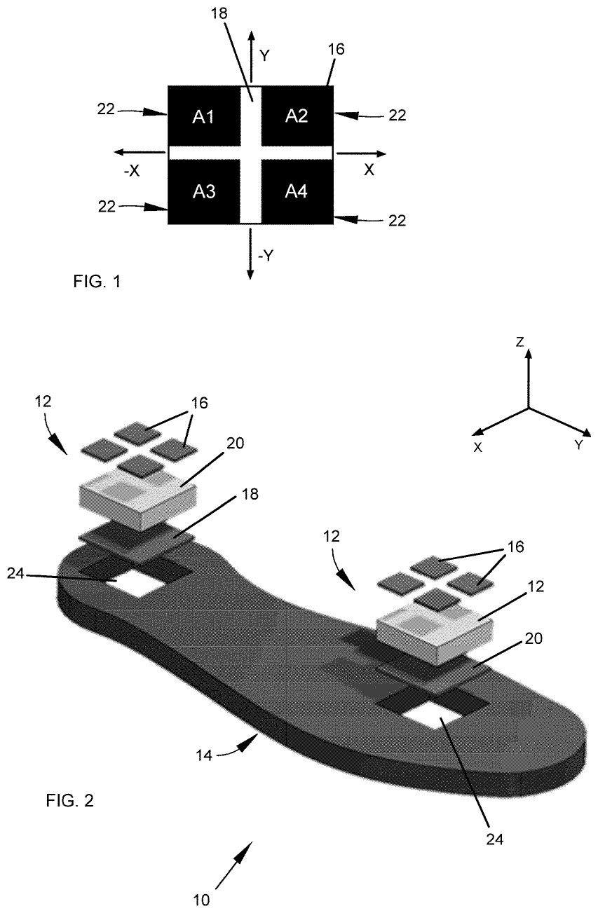

The sensors utilize capacitive sensing elements in the form of conductive plates that can be applied or deposited on a wide variety of materials and calibrated using static techniques for compatibility with a wide range of athletic performance and a wide range of physical action. FIG. 1 schematically represents a 2×2 array of individual parallel-plate capacitive units 22, each comprising an upper plate 16 superimposed on a single lower plate 18. FIG. 1 also indicates a naming system that will be used herein to identify areas of the upper plates 16 superimposed on the lower plate 18 for each capacitor unit 22. The upper plates 16 are laterally spaced apart from each other in what is designated in FIG. 1 as an X-Y plane, and the upper plates 16 are spaced apart from the lower plate 18 in what is designated herein as the Z-axis.

FIG. 2 is an exploded view of what will be referred to herein as a sensor system 10, and is representative of test specimens manufactured to investigate and evaluate the invention. FIG. 2 schematically shows the system 10 as comprising two sensors 12 each configured for individual placement in a recess, cavity, or other suitable opening 24 in a shoe insole 14. In the investigations, the insole 14 was formed of silicone and conductive plates 16 and 18 of each sensor 12 were formed of a graphene-silicon composite. Each capacitor unit 22 of each sensor 12 comprises one of the four individual upper plates 16 superimposed on the lower plate 18 as represented in FIG. 1. FIG. 2 further shows each sensor 12 as comprising a pliable dielectric 20 that separates the upper plates 16 from the lower plate 18. Consistent with FIG. 1, the upper plates 16 are represented as laterally spaced apart from each other, and therefore electrically insulated from each other by adjacent surface regions of the dielectric 20. In the nonlimiting embodiments shown in the drawings, each of the upper and lower plates 16 and 18 has a quadrilateral peripheral shape (boundary), the upper plates 16 have identical shapes and areas, and the outer corner of each upper plate 16 is superimposed on one of the corners of the lower plate 18 such that all four upper plates 16 are entirely superimposed on the lower plate 18. By applying an electrical potential between each upper plate 16 and the lower plate 18, capacitive sensing of each sensor 12 can be based on the relative movements of individual upper plates 16 relative to the lower plate 18, causing a change in capacitance between the upper and lower plates 16 and 18 of each capacitor unit 22. In investigations leading to embodiments of the invention, a combination of silicone-graphene composite conductive plates 16 and 18 and a silicone dielectric 20 was chosen for use because silicone-graphene composite is able to bond to silicone and similarly deform in response to normal and shear stresses. Though dissimilar conductive and dielectric materials could be used to construct the plates 16 and 18, deformation characteristics would presumably be affected.

Manufacturing processes for producing the sensors 12 and incorporating the sensors 12 into footwear are capable of allowing for a large range of adaptability. Various physical parameters of the sensors 12 can be modified, including the footprint of a sensor 12, the area of the upper and lower plates 16 and 18, and the type and thickness of the materials used to form the plates 16 and 18 and dielectric 20. The use of different densities and materials for the dielectric 20 allows for a customized force regime. Additionally, sensors 12 can be distributed throughout the insole 14 in any desired configuration. All of these parameters can be optimized to fit a desired application.

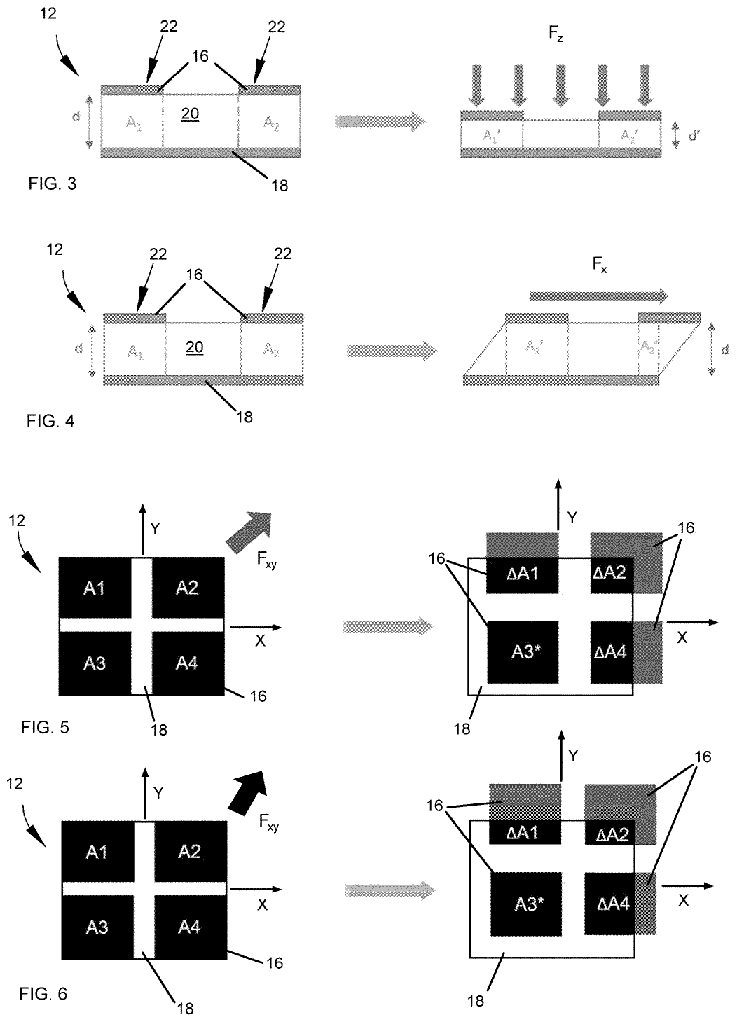

In the 3-D coordinate system used to characterize the sensors 12, normal forces act along the Z-axis with positive forces acting upward, and shear forces act within the X-Y plane approximately corresponding to the plane of the foot. As represented in FIG. 3, a normal force (F_{z}) results in deformation (compression) of the dielectric 20, causing a decrease in the distance (from d to d′) between the lower plate 18 and each of the upper plates 16 that corresponds to an increase in capacitance of an individual capacitor unit 22 according to the equation

c=Aε₀/d (EQ 1)

where c is capacitance, A is the area of an individual upper plate 16 that is superimposed on the lower plate 18, ε₀ is the permittivity of the material of the dielectric 20, and d is the distance between each upper plate 16 and the lower plate 18. As evident from FIG. 3 and EQ 1, when a capacitor unit 22 is subjected to a normal force, the distance (d) is the principal variable that determines capacitance (c).

As represented in FIG. 4, shear forces also result in deformation of the dielectric 20, associated with the lateral movement of one or more of the upper plates 16 relative to the lower plate 18 in the X-Y plane. As evident from FIG. 4, when a capacitor unit 22 is subjected to a shear force, the distance (d) and superimposed areas (A) are both variables that determine capacitance (c), the latter resulting from a portion of the area (A₂) of the righthand upper plate 16 not being entirely superimposed on the lower plate 18, while the remaining portion (A₂′) of the righthand upper plate 16 remains superimposed on the lower plate 18 to define a superimposed area therebetween. There are two possible shear forces that can be applied to a sensor 12: a “single-directional” force (Fₓ) shown in FIG. 4 as acting solely along the X axis (or, alternatively, the Y axis), or a “multidirectional” force (F_{xy}) shown in each of FIGS. 5 and 6 as acting in the X-Y plane but not along the X or Y axis. Shear forces are solved for by relating the change in superimposed area (from A to A′) of each capacitor unit 22 to the applied force in that direction. According to a particular aspect of the invention, relationships are ascertained between capacitor units 22 of a sensor 12 to determine in which direction a shear force is acting, and changes in superimposed area (from A to A′) determine the magnitude of the shear force. Before relationships can be ascertained, the normal force must be found as any change in the thickness of the dielectric 20 within a capacitor unit 22 (corresponding to a change in distance from d to d′) will alter the capacitance of the unit 22. Consequently, if the effect of a normal force is not accounted for, the change in superimposed area (from A to A′) used to calculate shear forces will be inaccurate. For the single-directional shear force (Fₓ) shown in FIG. 4, the two visible capacitor units 22 will exhibit a positive value for the change in their respective superimposed areas (from A₁ to A₁′, and from A₂ to A₂′). As seen from FIG. 4, the change in superimposed area is quantified by how much of the upper plate 16 of the righthand unit 22 remains superimposed on the lower plate 18.

For the multidirectional shear forces (F_{xy}) shown in FIGS. 5 and 6, the ascertainment of relationships between capacitor units 22 of a sensor 12 to determine in which direction the shear force is acting requires several steps. In FIG. 5, because the upper plate 16 of the capacitor unit 22 associated with area A3 remains entirely superimposed on the lower plate 18 and therefore exhibits no change in superimposed area, the capacitor unit 22 associated with area A3 is used as a basis to determine which direction the multidirectional shear force (F_{xy}) is acting. The multidirectional force is broken down into its two force components (Fₓ and F_{y}) to determine which unit 22 exhibits a change in superimposed area (ΔA) in both force components (the capacitor unit 22 associated with area A2 in FIG. 5), and that change in superimposed area is then quantifiably removed to quantify the change in superimposed area of only the remaining two units 22 (associated with areas A1 and A4), whose changes in superimposed area (ΔA1 and ΔA4) are then used to finally calculate the two force components (Fₓ and F_{y}) of the shear force (F_{xy}) in each single axis direction (X-axis and Y-axis). A comparison of FIGS. 5 and 6 evidences that the difference in the superimposed areas A1 and A4 correlates to the direction that the shear force (F_{xy}) is acting.

The sensors 12 can be connected to circuitry for analyzing their outputs. As a nonlimiting example, the sensors 12 can be wired to a MyRIO microcontroller board (National Instruments Corporation) and a battery, both of which may be located on a unit worn by the user. The outputs of the sensors 12 can then be read on the MyRIO microcontroller board using appropriate software, for example, LabVIEW software (National Instruments Corporation). LabVIEW can be used to pulse a current to the lower plate 18 of each capacitor unit 22, and use these pulses to read changing capacitive values as each sensor 12 is deformed. Referring again to FIG. 2, if a normal force (F_{z}) acts on a sensor 12, the distance (d) between the parallel upper and lower plates 16 and 18 will decrease, resulting in an increase in the capacitance of each capacitor unit 22 of the sensor 12. The increases in capacitance can then be read through the LabVIEW program as a decrease in voltage. Through appropriate calibration, voltage corresponds to force values sensed by the sensors 12. An average change in voltage measured across all four capacitor units 22 can be correlated to the application of the normal force represented in FIG. 2. During evaluations of the invention, “I2C” (inter-integrated circuit) communication was used to plot voltage data in real-time.

During investigations leading to the present invention, specimens were constructed and calibrated for use in evaluating injury prevention/sports performance applications. Sensors 12 of the type shown in FIG. 2 were wired to a MyRIO board and a battery, both of which were located on a unit worn around the waist of the user. The sensors 12 were tested statically and dynamically. As static calibration of force sensing technology is the current industry standard, a static rig was created to apply shear and normal forces to the sensors 12 on a force plate. Loading and unloading cases were applied in increments of five pounds (about 2.3 kg) to a total of thirty pounds (about 13.6 kg). The static capacitive sensor data was compared with the static force plate data for calibration. Dynamic testing was performed to assess how the sensors would withstand high impact dynamic loading. Dynamic testing was qualitative as the tests were only used to compare peaks in the force plate data to peaks in the data collected from sensors 12 located in the insole 14. After calibration was completed, exponential curves were used to determine necessary relationships. Voltages and the changes in superimposed area were compared for shear forces and the voltages and changes in distance between the plates 16 and 18 were compared for normal forces. This trend resulted in equations used to solve for the normal and shear forces. Correlations of at least 90 percent were discovered between exponential curve fits of shear force data from the sensors 12 as compared to their force-plate counterparts, which was concluded to be a successful showing of the accuracy and capabilities of the sensors 12.

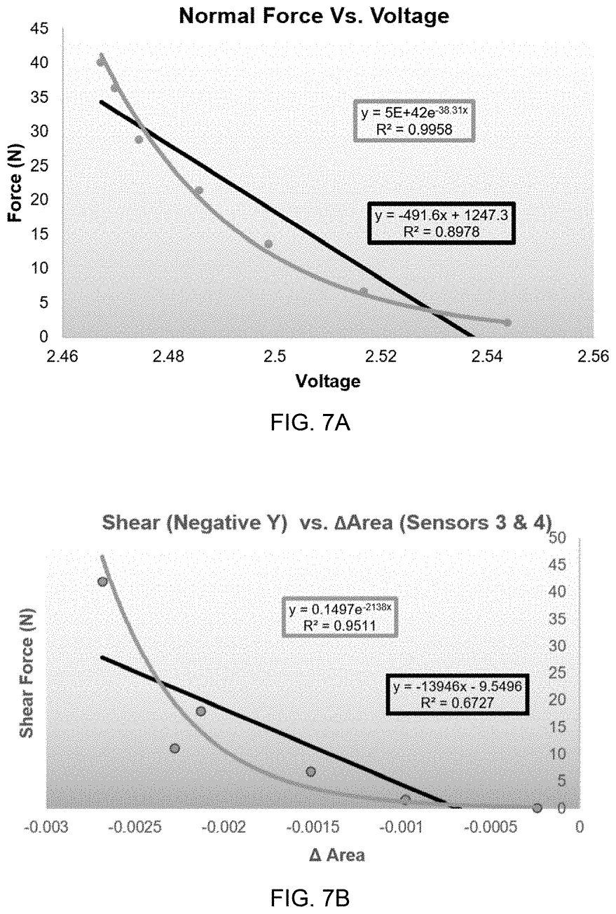

Representative calibration results shown in FIG. 7A evidence that there was a very strong correlation between an applied normal force and deformation in the normal (Z) direction. The same can be said for shear force and deformation in the X and Y directions. The relationships between force and deformation all followed an exponential curve with R∧2 values above 0.95 for multiple tests, as shown in FIG. 7B, evidencing an extremely strong correlation that enables normal and shear forces to be accurately measured with the sensors 12.

In a dynamic environment, calibration is adjusted to the ambiguity of direction within the application of a force. As noted above, calibration involves isolating which one of the four upper plates 16 is not associated with a change in its area superimposed on the lower plate 18. This isolation enables the measurement of any change in vertical distance (d) experienced by the capacitor units 22 of a sensor 12 to be determined, since any change in capacitance (c) of a unit 22 that does not experience a change in superimposed area (A) will be attributable only to a change in distance. A measured change in distance can then be applied throughout all four capacitor units 22 to determine the changes in superimposed area for the other units 22.

As a nonlimiting example of the above, an iterative technique can be utilized, for example:

F=k(Δd)

(d+Δd)=εA/C

Δd=εA/(Q/V)−d

Δd=VεA/IT−d (EQ 2)

This first derivation is for a normal force associated with the system and determines how a normal force (F_{z}) impacts the change in distance (d). The final equation from this derivation includes the relative permittivity (ε) of the dielectric material, the unchanged cross-sectional area of the material (A), the charge current (I), the charge time (T), and the unchanged distance (d) between the capacitive plates 16 and 18. The voltage here is the ambiguous part, because the voltage of the unit(s) 22 that has (or have) not changed in cross-sectional area must be determined for the equation to be valid. An iterative technique to find this unit 22 can involve an initial estimate, in which an average the voltages of all four units 22 are used to determine an initial estimate of the change in distance between the lower plate 18 and each upper plate 16. An estimated change in cross-sectional areas across the capacitive plates 16 and 18 can then be solved for. This can be found from the derivation below:

A₁=Q(d+Δd)/V₁ε

A₁=IT(d+Δd)/V₁ε,A₂A₃,A₄ (EQ 3)

With this derivation, the individual areas for each of the four capacitive upper plates 16 can be determined utilizing the change in distance estimate previously determined. A difference between each of the area calculations is the voltages that are input, with each voltage pairing with the area in question. Once these areas are found, the unit 22 having an area closest to its original area can be determined. The change in distance equation (EQ 1) can then be used to establish a change in distance for the entire sensor 12, which is then used in the area calculations for the remaining units 22. Once those areas are calculated, the changes in area can be determined to create relationships.

While the invention has been described in terms of a particular embodiment and particular investigations, it should be apparent that alternatives could be adopted by one skilled in the art. For example, the sensors 12 and their components could differ in appearance and construction from the embodiment described herein and shown in the drawings, functions of certain components of the systems 10 could be performed by components of different construction but capable of a similar (though not necessarily equivalent) function, and appropriate materials could be substituted for those noted. As a particular example, the sensors 12 could be configured to wirelessly communicate with appropriate processing means. As such, it should be understood that the above detailed description is intended to describe the particular embodiment represented in the drawings and certain but not necessarily all features and aspects thereof, and to identify certain but not necessarily all alternatives to the represented embodiment and described features and aspects. As a nonlimiting example, the invention encompasses additional or alternative embodiments in which one or more features or aspects of the disclosed embodiment could be eliminated. Accordingly, it should be understood that the invention is not necessarily limited to any embodiment described herein or illustrated in the drawings, and the phraseology and terminology employed above are for the purpose of describing the illustrated embodiment and investigations and do not necessarily serve as limitations to the scope of the invention. Therefore, the scope of the invention is to be limited only by the following claims.

1. A sensor for measuring normal and shear forces, the sensor comprising:
a first plate; and
multiple second plates separated from the first plate by a dielectric material to define multiple capacitor units that are each responsive to normal and shear forces applied to the sensor and each comprise an individual second plate of the second plates and a portion of the first plate that is superimposed by the individual second plate, the second plates comprising at least four second plates that are entirely superimposed on the first plate, wherein:
any shear force applied to the sensor causes a first portion of at least two of the four second plates to not be superimposed on the first plate while a remaining portion of each of the at least two of the four second plates remains superimposed on the first plate to define a superimposed area therebetween; and
all of at least one of the four second plates remains entirely superimposed on the first plate in response to any shear force applied to the sensor and therefore the at least one of the four second plates exhibits no change in a superimposed area between the first plate and the at least one of the four second plates as a basis to determine which direction the shear force acts on the sensor; and
which of the four second plates remains entirely superimposed on the first plate depends on the direction of the shear force acting on the sensor;
wherein each of the four second plates has an outer corner extremity and two peripheral edge extremities contiguous therewith that are, in absence of a shear force, individually superimposed on and thereby vertically aligned with a corresponding one of a plurality of outer corner extremities of the first plate and two peripheral edge extremities of the first plate that are contiguous therewith such that all of the four second plates are entirely superimposed on the first plate.

2. The sensor according to claim 1, wherein the second plates are superimposed on the first plate so that a normal force applied to the sensor is measured based on a change in distance between one or more of the second plates and the portion of the first plate superimposed by the at least one second plate.

3. The sensor according to claim 2, wherein the second plates are superimposed on the first plate so that a shear force applied to the sensor is measured based on a change in the superimposed area of the at least two of the four second plates relative to the first plate.

4. The sensor according to claim 1, wherein the second plates are superimposed on the first plate so that a shear force applied to the sensor is measured based on a change in the superimposed areas of the at least two of the four second plates relative to the first plate.

5. The sensor according to claim 1, wherein the second plates consist of the four second plates.

6. The sensor according to claim 1, wherein each of the first and second plates has a quadrilateral peripheral boundary and the four second plates have identical shapes and areas.

7. The sensor according to claim 1, wherein the sensor is a component of a sensing system that comprises apparel in which the sensor is embedded.

8. The sensor according to claim 7, wherein the apparel is a shoe.

9. The sensor according to claim 7, wherein the sensor is one of a plurality of the sensor embedded in the apparel.

10. The sensor according to claim 7, wherein the second plates are superimposed on the first plate so that a normal force applied to the sensor is measured based on a change in distance between one or more of the second plates and the portion of the first plate superimposed by the at least one second plate.

11. The sensor according to claim 10, wherein the second plates are superimposed on the first plate so that a shear force applied to the sensor is measured based on a change in the superimposed area of the at least two of the four second plates relative to the first plate.

12. The sensor according to claim 7, wherein each of the first and second plates has a quadrilateral peripheral boundary and the four second plates have identical shapes and areas.

13. A method of using the sensor of claim 1, the method comprising:
embedding the sensor in apparel;
a user wearing the apparel while performing a physical activity; and
measuring with the sensor normal and shear forces to which the user is subjected as a result of the physical activity, and using the superimposed area between the first plate and the at least one of the four second plates as a basis to determine the direction that the shear force acts on the sensor.

14. The method according to claim 13, wherein the apparel is a shoe and the sensor measures normal and shears forces to which a foot of the user is subjected as a result of the physical activity.

15. A sensing system comprising:
apparel; and
a sensor embedded in the apparel for measuring normal and shear forces to which a user wearing the apparel is subjected, the sensor comprising a first plate and multiple second plates separated from the first plate by a dielectric material to define multiple capacitor units that are each responsive to normal and shear forces applied to the sensor and each comprise an individual second plate of the second plates and a portion of the first plate that is superimposed by the individual second plate, the second plates comprising at least four second plates that are entirely superimposed on the first plate, wherein:
any shear force applied to the sensor causes a first portion of at least two of the four second plates to not be superimposed on the first plate while a remaining portion of each of the at least two of the four second plates remains superimposed on the first plate to define a superimposed area therebetween; and
all of at least one of the four second plates remains entirely superimposed on the first plate in response to any shear force applied to the sensor and therefore the at least one of the four second plates exhibits no change in a superimposed area between the first plate and the at least one of the four second plates as a basis to determine which direction the shear force acts on the sensor; and
which of the four second plates remains entirely superimposed on the first plate depends on the direction of the shear force acting on the sensor;
wherein each of the four second plates has an outer corner extremity and two peripheral edge extremities contiguous therewith that are, in absence of a shear force, individually superimposed on and thereby vertically aligned with a corresponding one of a plurality of outer corner extremities of the first plate and two peripheral edge extremities of the first plate that are contiguous therewith such that all of the four second plates are entirely superimposed on the first plate.
